(19) United States
(12) Patent Application Publication
(10) Pub. No.: US 2008/0223949 A1
(43) Pub. Date: Sep. 18, 2008

(54) Wireless remote controlled air freshening and sound generating machine

(76) Inventor: Shawn Sweeney, Upper Saddle River, NJ (US)

Correspondence Address: Shawn Sweeney, 17 Cottage Lane, Upper Saddle River, NJ, 07458, US

(21) Appl. No.: 11/717,351

(22) Filed: Mar. 13, 2007

(51) Int. Cl. A61L 9/04

(52) U.S. Cl. 239/34

(57) ABSTRACT

A Remote controlled air freshening and sound generating machine with a radio control transmitter, a radio controlled receiver, an internal speaker, an internal battery compartment accessible from the outer housing, a canister of scent, a mechanical actuator, and switches to allow sound or scent activation or deactivation individually, a circuit board containing the preprogrammed sounds, a button allowing activation on the unit itself, and a self containing outer housing, and an aperture in the outer housing to allow the dispersing of scent.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of air freshening and more specifically to a wireless remote controlled battery powered air freshening and sound generating machine.

Fresh smelling air is a quality of life concern. Often situations occur where an instantaneous, on demand freshening of the air is needed. The new technology is inspired from the need to dispense air freshening scents whenever, and wherever it is needed via remote control while accompanied by an appropriate preprogrammed sound. There is no previous technology which can provide this combination of features in one contained unit.

### PRIOR TECHNOLOGY EXISTS IN PATENTS

D250,042 Pardo; John (Yonkers, N.Y.), Jones; David A. (Kettering, Ohio) air freshening device which constantly freshens air with no controls to user D515,192 Smith; Doreen (South Deerfield, Mass.), Lenhart; Vikki (Amherst, Mass.), Shepherd; Rebecca J. (Mentone, Ind.), Wolf; Lawrence R. (Avilla, Ind.) air freshening device which is both constantly on and not portable

U.S. Pat. No. 5,273,690 McDowell; John L. (Sun Valley, Calif.) air freshening device dependent on forced air to operate

U.S. Pat. No. 6,123,935 Wefler; Mark E. (Mount Pleasant, Wis.), Martin; John (Caledonia, Wis.), D250,042 Pardo; John (Yonkers, N.Y.), Jones; David A. (Kettering, Ohio), D364,919 Adams; Mary B. (Gurnee, Ill.), do not allow for instantaneous activation via wireless remote control in the event of sudden odor in the vicinity of the unit. Prior technology is also deficient in that it sometimes dispenses scent whether needed or not which is wasteful of the scent. The new technology compensates for all of these deficiencies.

Prior technology allows for air fresheners that constantly dispense scent, or via preprogrammed intervals, or require hands on use. It may dispense even when it may not be needed. This use of scent with prior technology can be wasteful and not economical. Prior technology may not contain an appropriate sound when dispensed either.

The benefit of the new technology is to allow for the dispensing of scent only when the user deems necessary and delivers an appropriate sound if the user so desires. It is also completely self contained and portable which means it can be placed anywhere air freshening may be needed. Once the unit is placed the user need not be near the unit in order to activate it via remote control. It can also be activated by hands on use of the primary unit. The unit is not only functional but can be decorated in unlimited design by applications of decals and by using different colored plastics for its outer cover. Furthermore a myriad of appropriate sounds and scents can be used for any use or aplication conceivable.

### BRIEF SUMMARY OF THE INVENTION

The primary object of the invention is to provide a remote controlled air freshening device activated via remote control or via activation on the unit itself.

Another object of the invention is to provide appropriate sounds via speaker with the dispensing of scent.

Another object of the invention is to provide a completely wireless air freshening device.

A further object of the invention is to provide an air freshening device that can be placed in needed areas regardless of household electrical availability.

Yet another object of the invention is to provide an air freshening device that only uses its contained scent when deemed necessary by the user.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a remote controlled air freshening and sound generating machine comprising: a remote controlled transmitter, an internal speaker, an internal battery compartment with external access, a canister of scent with external access, a mechanical piston, a circuit board on which are mounted the remote controlled receiver, externally accessible switches to allow sound or scent activation or deactivation individually, unit power, preprogrammed sounds, a button allowing activation on the unit itself, and a self containing outer housing.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a plan view of the invention rear.

FIG. 2 is a plan view of the invention front including the remote controlled transmitter.

FIG. 3 is a plan view of the invention front without cover.

FIG. 4 is a plan view of the invention rear without cover.

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

### DETAILED DESCRIPTION OF THE EMBODIMENTS

The present invention is a device that freshens the air and plays appropriate sounds when doing so. The user when in need of freshly scented air and or a pleasing sound need only press the button on the wireless remote control or press the button on the unit itself.

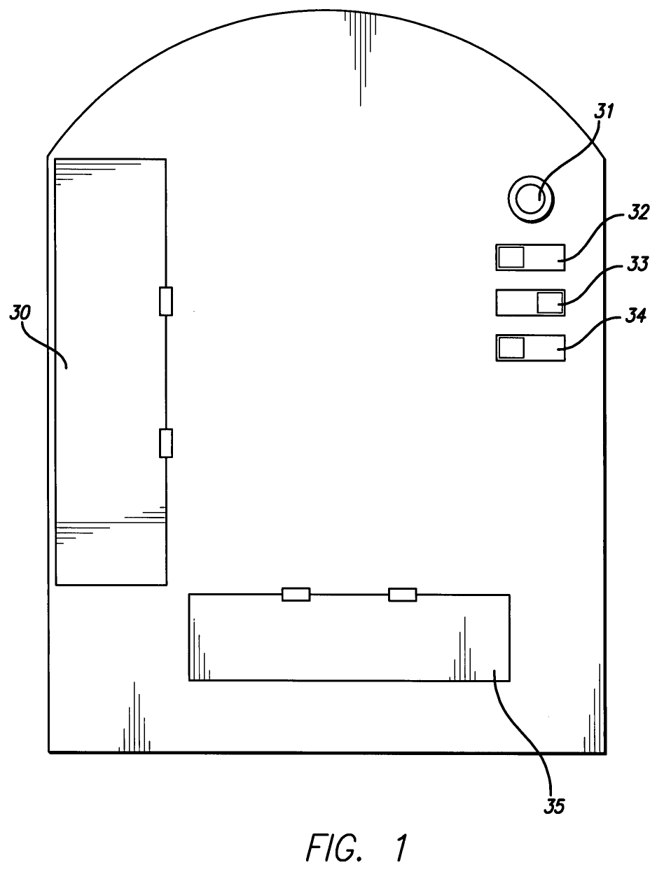

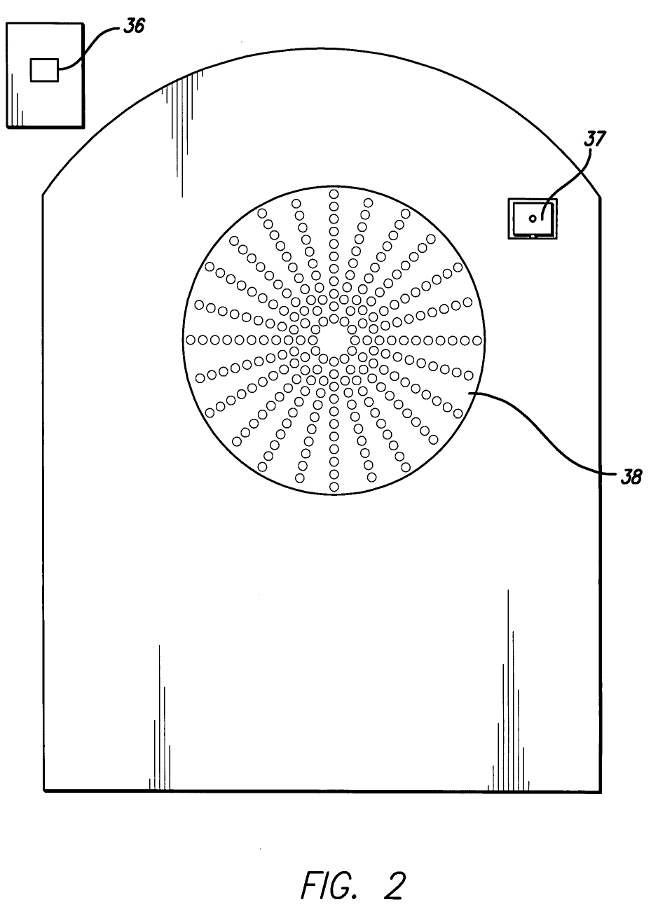

FIG. 1 (rear of unit) shows a door 30 allowing access to the scent canister. The user can open the door 30 to change the canister when needed. Another door 35 allows access to the battery compartment. The user can open the door 35 and change the batteries when needed. The rear has a button for activation 31; and 3 switches. A switch which turns sound on and off 32, a switch which turns scent on and off 33, and a switch which powers the unit on and off 34. Once power is turned on via switch 34, the user will activate the unit by either pressing button 31 on the unit itself, or by pressing the button on the wireless remote control 36 on FIG. 2. The user will then receive unit performance desired in accordance with the positions of switches sound 32 and/or scent 33.

FIG. 2 (front of unit) shows an aperture in the cover 37, through which scent is dispensed. The cover also shows a perforated area 38, which allows the sound to be heard. Also shown in FIG. 2 is the battery powered remote controlled transmitter with button 36. The invention activates when the button 36 is pressed on the wireless remote control.

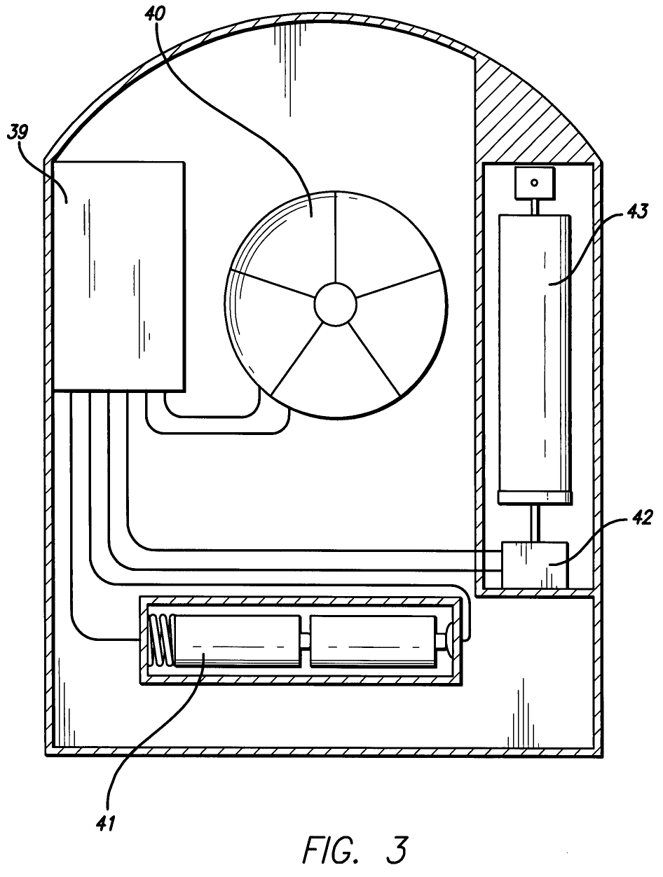

FIG. 3 (front of unit without cover) shows the backside of the circuit board 39. It also shows the speaker front 40, and the battery compartment 41. In addition it also shows the scent canister 43, as well as the mechanical piston which presses upward against the scent canister when activated to dispense scent.

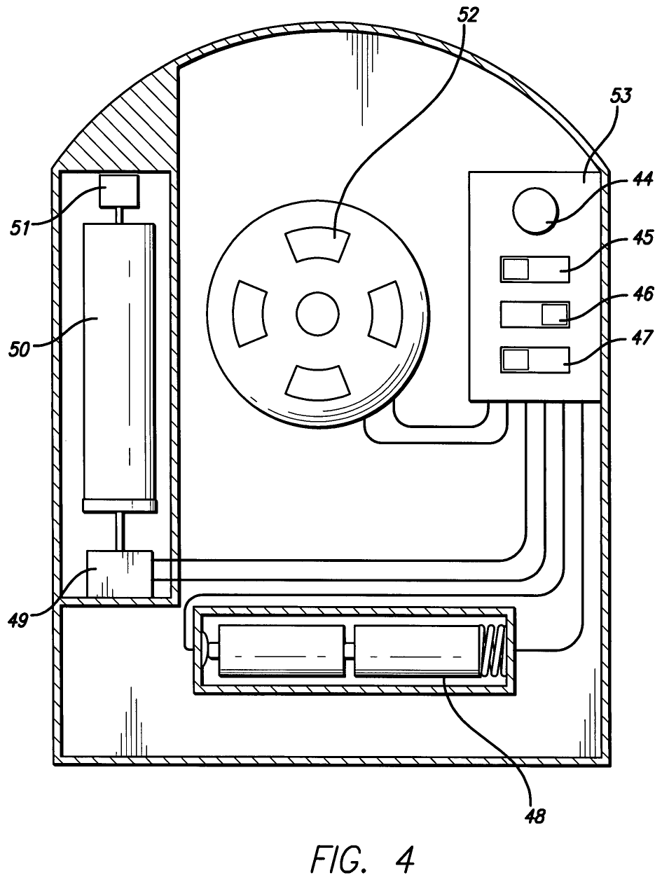

FIG. 4 (rear of unit without cover) shows the mechanical piston 49, the scent canister 50, and the dispensing tip 51. In addition it shows the speaker rear 52 and battery compartment 48. The circuit board 53 has the remote controlled transmitter's receiver, a button for activation 44; a switch which turns sound on and off 45, a switch which turns scent on and off 46, and a switch which powers the unit on and off 47. The circuit board 53 also contains the preprogrammed sound. When the unit is activated via remote control button 36 (FIG. 2) or via button 44 the unit delivers scent by activating the piston 49 below the scent canister and compressing its tip then resetting allowing a short burst of scent to escape through the aperture 37 (FIG. 2) in the front cover. Upon activation the unit will also simultaneously play its preprogrammed sound stored on the circuit board 53, through the internal speaker 52. If the user decides to use the unit without sound or without scent this can be accomplished by disabling either function via switches 45 and 46 before activation.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A Remote controlled air freshening and sound generating machine comprising:
A radio control transmitter;
A radio controlled receiver;
An internal speaker;
An internal battery compartment;
A canister of scent;
A mechanical piston;
Switches to allow sound or scent activation or deactivation individually;
A circuit board containing the preprogrammed sounds buttons, switches and receiver for wireless transmitter;
A button allowing activation on the unit itself; and
A self containing outer housing.
